(19) United States
(12) Patent Application Publication
(10) Pub. No.: US 2004/0077621 A1
(43) Pub. Date: Apr. 22, 2004

(54) Antioxidants

(75) Inventor: Chi-Ming Yang, Taipei County (TW)

Correspondence Address: COHEN, PONTANI, LIEBERMAN & PAVANE, Suite 1210, 551 Fifth Avenue, New York, NY, 10176, US

(73) Assignee: Academia Sinica

(21) Appl. No.: 10/637,770

(22) Filed: Aug. 8, 2003

Related U.S. Application Data
(60) Provisional application No. 60/401,898, filed on Aug. 8, 2002.

(51) Int. Cl. A61K 31/555; A61K 31/409

(52) U.S. Cl. 514/185; 514/410

(57) ABSTRACT

This invention features a method for eliciting an antioxidative effect. The method includes administering to a subject (e.g., an animal or human) in need thereof an effective amount of a tetrapyrrole compound of formula (I):

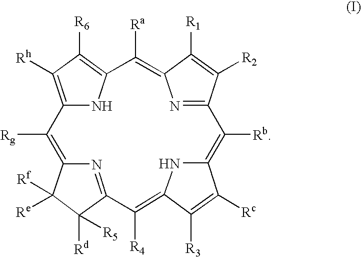

Each of R^{a}, R^{b}, R^{c}, R^{d}, R^{e}, R^{f}, R^{g}, and R^{h}, independently, is H or alkyl; R₁ is alkyl or CHO; R₂ is alkyl; R₃ and R₄ taken together are part of a cyclyl or heterocyclyl ring, and R₅ is (CH₂)ₘCOOR, in which R is H, and m is 1, 2, or 3; or R₃ is (CH₂), COOR′, C(O)(CH₂)ₙCOOR′, CH(OH)(CH₂)ₙCOOR′, and R₄ and R₅ taken together are part of a cyclyl or heterocyclyl ring, in which R′ is H or alkyl, and n is 0, 1, 2, or 3; and R₆ is alkenyl or CHO; the tetrapyrrole compound optionally being chelated with Mg²⁺, Mn²⁺, Cu²⁺, Fe²⁺, Co²⁺, Ni²⁺, or Zn²⁺ through the nitrogen atoms on the four pyrrole rings.

### CROSS REFERENCE TO RELATED APPLICATIONS

This application claims priority to U.S. Provisional Application Serial No. 60/401,898, filed on Aug. 8, 2002, the contents of which are incorporated herein by reference.

### BACKGROUND OF THE INVENTION

### BACKGROUND

Chlorophylls are the green photosynthetic pigments present in chloroplasts (organelles in plant and eukaryotic algae cells). They are capable of channeling the energy of sunlight into chemical energy through the process of photosynthesis. In this process, the energy absorbed by chlorophylls transforms carbon dioxide and water into carbohydrates and oxygen. See, e.g., Battersby (1985) Pro. R. Soc. Lond. B. 225: 1-26; and Rudiger & Schoch (1988) Chlorophylls. In Plant Pigments. Goodwin, T. W. (ed) pp.1-59. Academic Press, London. In the thylakoid membrane of chloroplasts, chlorophylls are non-covalently bound to specific intrinsic polypeptides as pigment-protein complexes. They are biosynthesized from glutamate, and degraded into small molecules. See, e.g., Markwell et al. (1979) Proc. Natl. Acad. Sci. USA 76: 1233-1235; Matile et al. (1996) Plant Physiol. 112: 1403-1409; and Reinbothe & Reinbothe (1996) Plant Physiol. 111: 1-7. The function of chlorophylls in plant photosynthesis is well understood; however, questions remain as to their biological functions in animals and humans.

### SUMMARY

The present invention is based, in part, on the discovery that chlorophyll analogs are capable of eliciting an antioxidative effect in animals or humans.

In one aspect, this invention features a method for eliciting an antioxidative effect. The method includes administering to a subject (e.g., an animal or human) in need thereof an effective amount of a tetrapyrrole compound of formula (I):

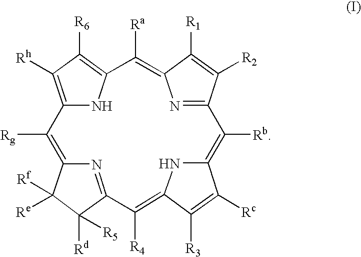

Each of R^{a}, R^{b}, R^{c}, R^{d}, R^{e}, R^{f}, R^{g}, and R^{h}, independently, is H or alkyl; R₁ is alkyl Or CHO; R₂ is alkyl; R₃ and R₄ taken together are part of a cyclyl or heterocyclyl ring, and R₅ is (CH₂)ₘCOOR, in which R is H, and m is 1, 2, or 3; or R₃ is (CH₂)ₙCOOR′, C(O)(CH₂)ₙCOOR′, CH(OH)(CH₂)ₙCOOR′, and R₄ and R₅ taken together are part of a cyclyl or heterocyclyl ring, in which R′ is H or alkyl, and n is 0, 1, 2, or 3; and R₆ is alkenyl or CHO; the tetrapyrrole compound optionally being chelated with Mg²⁺, Mn²⁺, Cu²⁺, Fe²⁺, Co2+, Ni²⁺, or Zn2+ through the nitrogen atoms on the four pyrrole rings. Note that the left-most atom shown in any of the substituted groups described above is the one closest to the four pyrrole rings.

One subset of these tetrapyrrole compounds is one in which each of R^{a}, R^{b}, R^{d}, R^{e}, and R^{g} is H; and each of R^{c}, R^{f} and R^{h} is CH₃. Another subset contains compounds wherein the tetrapyrrole compound is chelated with Mg²⁺ through the nitrogen atoms on the four pyrrole rings. A third subset includes compounds wherein R₂ is CH₂CH₃ and R₁ is CH₃ or

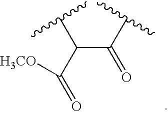

CHO; or R₃ and R₄ taken together are

Still another subset of the tetrapyrrole compounds are those wherein R₅ is CH₂CH₂COOH, and R₆ is CH₂═CH₂. In these compounds, each of R^{a}, R^{b}, R^{d}, R^{e}, and R^{g} can be H; each of R^{c}, R^{f} and R^{h} can be CH₃; the tetrapyrrole compound can be chelated with Mg²⁺ through the nitrogen atoms on the four pyrrole rings; R₁ can be CH₃ or CHO; R₂ can be CH₂CH₃; and R₃ and R₄ taken together can be

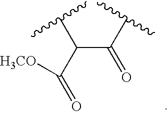

CH₂CH₃; and R₃ and R₄ taken together can be

In another aspect, this invention features a method for treating cancer. This method includes administering to a subject in need thereof an effective amount of a tetrapyrrole compound of formula (I), wherein each of R^{a}, R^{b}, R^{c}, R^{d}, R^{e}, R^{f}, R^{g}, and R^{h}, independently, is H or alkyl; R₁ is alkyl or CHO; R₂ is alkyl; R₃ and R₄ taken together are part of a cyclyl or heterocyclyl ring, and Rs is (CH₂)ₘCOOR, in which R is H, or [CH2—CH═C(CH₃)—CH₂]ₘ₁-[CH₂—CH₂—CH(CH₃)—CH₂]ₘ₂-[CH₂—CH═C(CH₃)—CH₂]ₘ₃-[CH₂—CH₂—CH(CH₃)—CH₂]ₘ₄—H; m is 1, 2, or 3, and each of m1, m2, m3, and m4, independently, is 0, 1, 2, 3, 4, or 5; or R₃ is (CH₂)ₙCOOR′, C(O)(CH₂)ₙCOOR′, CH(OH)(CH₂)ₙCOOR′, and R₄ and R₅ taken together are part of a cyclyl or heterocyclyl ring, in which R′ is H or alkyl, and n is 0, 1, 2, or 3; and R₆ is alkenyl or CHO; provided that if R is H, the tetrapyrrole compound is optionally chelated with Mg²⁺, Mn²⁺, Cu²⁺, Fe²⁺, Co²⁺, Ni²⁺, or Zn²⁺ through the nitrogen atoms on the four pyrrole rings.

A subset of the above class of tetrapyrrole compounds is one in which R₅ is (CH₂)ₘCOO[CH₂—CH═C(CH₃)—CH₂]ₘ₁-[CH₂—CH₂—CH(CH₃)—CH₂]ₘ₂—[CH₂—CH═C(CH₃)—CH₂]ₘ₃—[CH₂—CH₂—CH(CH₃)—CH₂]ₘ₄—H, wherein m is 2, m1 is 1, m2 is 3, and each of m3 and m4 is 0. In these compounds, each of R^{a}, R^{b}, R^{d}, R^{e}, and R^{g} can be H; each of R^{c}, R^{f} and R^{h} can be CH₃; R₁ can be CH₃ or CHO; R₂ can be CH₂CH₃; R₃ and R₄ taken together can be

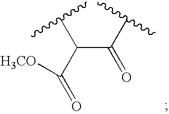

and R₆ can be CH═CH₂.

Another subset of the compounds are those wherein R₅ is (CH₂)ₘCOOH, and m is 2. In these compounds, each of R^{a}, R^{b}, R^{d}, R^{e}, and R^{g} can be H; each of R^{c}, R^{f} and R^{h} can be CH₃; R₁ can be CH₃ or CHO; R₂ can be CH₂CH₃; R₃ and R₄ taken together can be

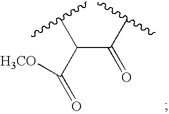

and R₆ can be CH═CH₂.

Alkyl, alkenyl, cyclyl, and heterocyclyl groups recited above include both substituted and unsubstituted moieties. The term “substituted” refers to one or more substituents (which may be the same or different), each replacing a hydrogen atom. Examples of substituents include halogen, hydroxyl, amino, cyano, nitro, mercapto, carbonyl, carbamido, carbamyl, carboxyl, thioureido, thiocyanato, sulfoamido, and alkyl.

As used herein, the term “alkyl” refers to a straight-chained or branched alkyl group containing 1 to 6 carbon atoms. Examples of alkyl groups include methyl, ethyl, n-propyl, isopropyl, and tert-butyl.

The term “alkenyl” refers to a straight-chained or branched alkenyl group containing 2 to 6 carbon atoms. Examples of alkenyl groups include vinyl, allyl (2-propenyl), dimethylallyl, and butenyl.

The terms “cyclyl” and “heterocyclyl” refer to partially and fully saturated mono- or bi-cyclic hydrocarbon ring systems having from 4 to 14 ring atoms. A heterocyclyl ring contains one or more heteroatoms (e.g., O, N, or S) as part of the ring. Exemplary cyclyl and heterocyclyl rings are cycylohexane, piperidine, piperazine, morpholine, thiomorpholine, and 1,4-oxazepane.

Below are exemplary compounds that can be used to practice methods of this invention:

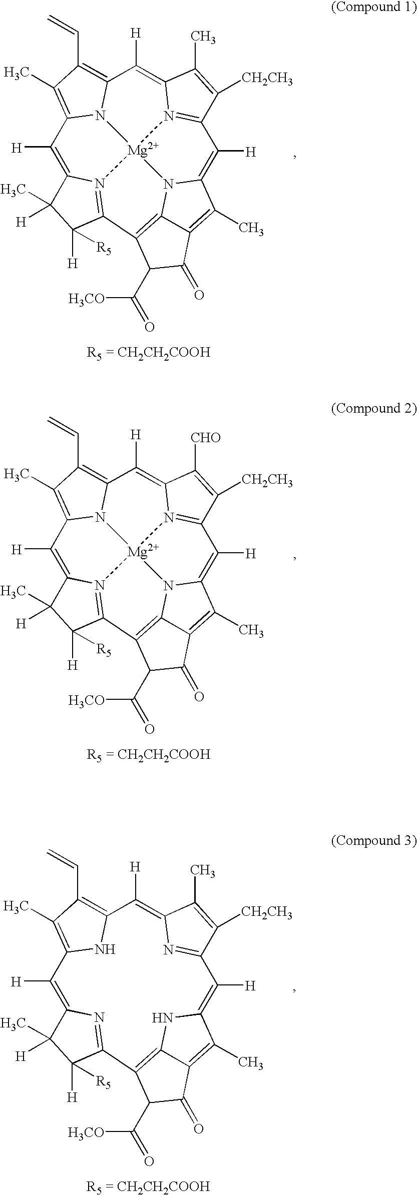

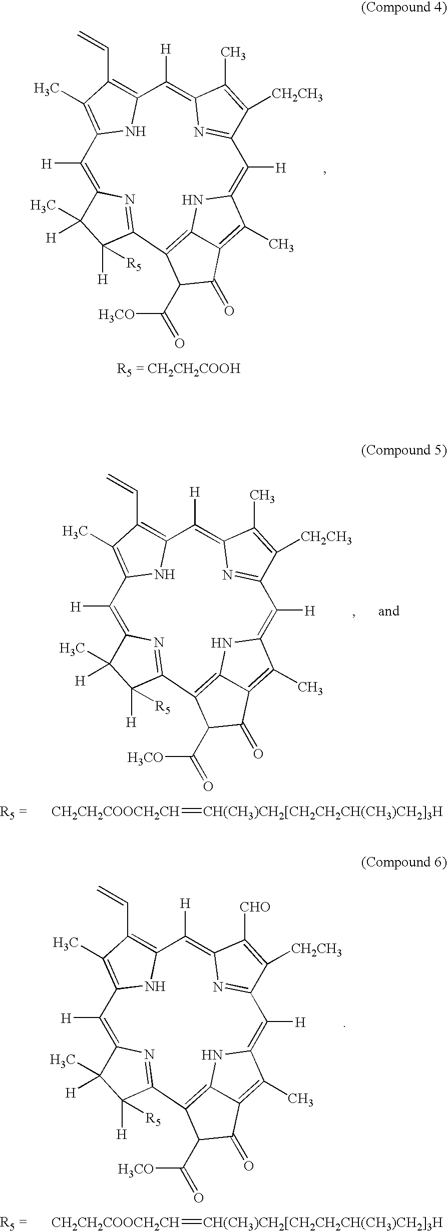

The tetrapyrrole compounds described above include the compounds themselves, as well as their salts and their prodrugs, if applicable. Such salts, for example, can be formed between a positively charged substituent (e.g., amino) on a compound and an anion. Suitable anions include, but are not limited to, chloride, bromide, iodide, sulfate, nitrate, phosphate, citrate, methanesulfonate, trifluoroacetate, and acetate. Likewise, a negatively charged substituent (e.g., carboxylate) on a compound can form a salt with a cation. Suitable cations include, but are not limited to, sodium ion, potassium ion, magnesium ion, calcium ion, and an ammonium cation such as teteramethylammonium ion. Examples of prodrugs include esters and other pharmaceutically acceptable derivatives, which, upon administration to a subject, are capable of providing the tetrapyrrole compounds described above.

In addition, some of the tetrapyrrole compounds have one or more double bonds, or one or more asymmetric centers. Such compounds can occur as racemates, tautomers, racemic mixtures, single enantiomers, individual diastereomers, diastereomeric mixtures, and cis- or trans- or E- or Z-double isomeric forms.

Further, the aforementioned tetrapyrrole compounds also include their N-oxides. The term “N-oxides” refers to one or more nitrogen atoms, when present in a compound, are in N-oxide form, i.e., N→O.

Also within the scope of this invention is a composition containing one or more of the tetrapyrrole compounds described above for use in eliciting an antioxidative effect in a subject, or in treating cancer, and the use of such a composition for the manufacture of a medicament for the just-described use.

Other features, objects, and advantages of the invention will be apparent from the description and from the claims.

### DETAILED DESCRIPTION

The tetrapyrrole compounds described above can be enriched from plants, and further modified by methods well known in the art. See, e.g., McFeeters et al. Pant Physol. (1971) 47:609-618 and Omata et al. Plant Cell Physiol. (1983) 24:1093-1100. For example, chlorophylls a and b can be purified from spinach, and dephytylated by the catalysis of chlorophyllase, which is isolated from plant Ficus macrocarpa leaf, to form Compounds 1 and 2, respectively. Compound 1, Compound 2, chlorophylls a, and chlorophylls b are magnesium(Mg)-dechelated by HCl to form Compounds 3-6, respectively. The tetrapyrrole comrpounds can also be prepared by synthetic methods well known in the art. Synthetic chemistry transformations and protecting group methodologies (protection and deprotection) useful in synthesizing applicable tetrapyrrole compounds are known in the art and include, for example, those described in Larock (1989) Comprehensive Organic Transformations, VCH Publishers; Greene & Wuts (1999) Protective Groups in Organic Synthesis, 3^{rd} Ed., John Wiley and Sons; Fieser & Fieser (1994) Fieser and Fieser's Reagents for Organic Synthesis, John Wiley and Sons; Paquette, ed. (1995) Encyclopedia of Reagents for Organic Synthesis, John Wiley and Son; and subsequent editions thereof.

The purity of the thus enriched or synthesized tetrapyrrole compounds can be readily measured by any appropriate method, for example, column chromatography, or high pressure liquid chromatography analysis. Further purification, if necessary, can be performed by methods well known in the art, such as high pressure liquid chromatography or recrystallization.

Also within the scope of this invention is a pharmaceutical composition that contains an effective amount of one or more of the tetrapyrrole compounds described in Summary and a pharmaceutically acceptable carrier. Further, the present invention covers a method of administering an effective amount of such a compound to a subject in need of eliciting an antioxidative effect or in need of treating cancer.

As used herein, a subject in need of eliciting an antioxidative effect can be a subject in need of anticlastogenicity, antimutagenicity, anticarcinogenicity, or antigenotoxicity treatment. See, e.g., Sarkar et al. Mutat. Res. (1994) 318:239-247 and Negishi et al. Mutat. Res. (1997) 376:97-100. The term “anti-oxidative effect” refers to the effect of protecting cells from oxidative damage (e.g., DNA oxidative damage) by reactive free radicals (e.g., oxygen free radicals). The term “treating” or “treatment” is defined as the application or administration of a composition including the aforementioned tetrapyrrole compound to a subject, who has a disease, a symptom of the disease, or a predisposition toward the disease, with the purpose to cure, heal, alleviate, relieve, alter, remedy, ameliorate, improve, or affect the disease, the symptoms of the disease, or the predisposition toward the disease. The term “cancers” refers to cellular tumor. Cancer cells have the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type, or stage of invasiveness. Examples of cancers include, but are not limited to, carcinoma and sarcoma such as leukemia, sarcomas, osteosarcoma, lymphomas, melanoma, ovarian cancer, skin cancer, testicular cancer, gastric cancer, pancreatic cancer, renal cancer, breast cancer, prostate colorectal cancer, cancer of head and neck, brain cancer, esophageal cancer, bladder cancer, adrenal cortical cancer, lung cancer, bronchus cancer, endometrial cancer, nasopharyngeal cancer, cervical or hepatic cancer, or cancer of an unknown primary site. In addition, cancer can be a drug resistance phenotype wherein cancer cells express P-glycoprotein, multidrug resistance-associated proteins, lung cancer resistance-associated proteins, breast cancer resistance proteins, or other proteins associated with resistance to anticancer drugs.

“An effective amount” refers to the amount of the tetrapyrrole compound which is required to confer a therapeutic effect on a treated subject. The interrelationship of dosages for animals and humans (based on milligrams per meter squared of body surface) is described in Freireich et al., (1966) Cancer Chemother Rep 50: 219. Body surface area may be approximately determined from the height and weight of a patient. See, e.g., Scientific Tables, Geigy Pharmaceuticals, Ardley, N.Y., 1970, 537. An effective amount of the compound described in Summary can range from about 175 mg/Kg to about 375 mg/Kg. Effective doses will also vary, as recognized by those skilled in the art, depending on the diseases treated, route of administration, excipient usage, and the possibility of co-usage with other therapeutic treatments such as use of other-agents.

To practice the method of the present invention, any of the tetrapyrrole compounds described above, as an active component of a pharmaceutical composition, can be administered parenterally, orally, by inhalation spray, topically, rectally, nasally, buccally, vaginally, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. Compounds described herein can also be administered in the form of suppositories for rectal administration. Implantable devices and related technology are known in the art and are useful as delivery systems where a continuous, or timed-release delivery of the compositions is desired. Additionally, the implantable device delivery system is useful for targeting specific points of composition delivery (e.g., localized sites or organs). Negrin et al (2001) Biomaterials 22(6): 563. Timed-release technology involving alternate delivery methods can also be used in this invention. For example, timed-release formulations based on polymer technologies, sustained-release techniques and encapsulation techniques (e.g., polymeric or liposomal) can also be used for delivery of the compositions.

The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form specific, more soluble complexes with the tetrapyrrole compounds delineated herein, or one or more solubilizing agents, can be utilized as pharmaceutical excipients for delivery of the tetrapyrrole compounds. Examples of other carriers include colloidal silicon dioxide, magnesium stearate, cellulose, sodium lauryl sulfate, and D&C Yellow # 10.

The biological activities (e.g., antioxidative activities) of tetrapyrrole compounds described above can be evaluated by a number of assays. For example, an antioxidative activity of a test compound is evaluated by determining the compound's ability to inhibit the formation of conjugated diene during topper-induced linoleic acid peroxidation (Wallin et al (1993) Anal. Biochem. 208: 10-15), or to inhibit the formation of malonyldialdhyde (MDA) during human low density lipoprotein (LDL) peroxidation (Yagi (1982) In lipid peroxides in Biology and Medicine. Yagi, K. (ed), pp. 223-242, Academic Press, New York). In another example, an antioxidative activity of a test compound is evaluated by determining the compound's ability to scavenge the free radicals of [symbol] -diphenyl- [symbol] -picrylhydrazyl (DPPH), or to scavenge superoxide anion, or to chelate Fe2+ cation, or to reduce power. See, e.g., Shimada et aL (1992) J. Agric. Food. Chem. 40: 945-948; Robak & Gryglewski (1988) Biochem. Pharma. 37: 837-841; Dinis et aL (1994) Arch Biochem Biophys 315: 161-169; and Oyaizu (1986) Nippon Shokuhin Kogyo Gakkaishi 35: 771-775.

Any of the tetrapyrrole compounds described above may be further evaluated by animal studies using methods well known in the art.

Without further elaboration, it is believed that the above description has adequately enabled the present invention. The following specific embodiments are, therefore, to be construed as merely illustrative, and not limitative of the remainder of the disclosure in any way whatsoever. All of the publications cited herein are hereby incorporated by reference in their entirety.

Antioxidative Activities of Compounds 1-6

Chlorophylls a and b were isolated from spinach, and dephytylated in the presence of a catalyst, chlorophyllase, which was isolated from plant Ficus macrocarpa leaf, to form Compounds 1 and 2, respectively. Compound 1, Compound 2, chlorophylls a, and chlorophylls b were magnesium(Mg)-dechelated by HCl to form Compounds 3-6, respectively.

Under a linoleic acid emulsion assay (see, e.g., Mitsuda et al. Eiyo Shokuryo (1966) 19:210-0.214 and Wallin et al. Anal. Biochem. (1993) 208:10-15), various degrees of inhibition of conjugated diene formation were observed during lipid peroxidation. In addition to Compounds 1-6, chlorophylls a and b were also tested. The results show that compounds with phytol chains, e.g., Compounds 5 and 6, had stronger antioxidative activity than those without phytol chains, e.g., Compounds 3 and 4. Further, compounds chelated with Mg, e.g., Compounds 1 and 2, had stronger antioxidative property than those without chelation, e.g., Compounds 3 and 4. The IC₅₀ values (i.e., the concentration required for 50% inhibition) were between 0.4 and 0.55 μM for Compounds 5 and 6, were between 1.0 and 1.2 μM for Compounds 1 and 2, and were higher than 1.4 μM for Compounds 3 and 4. Under a LDL measurement assay (see, e.g., Yagi (1982) In lipid peroxides in Biology and Medicine. Yagi, K. (ed), pp. 223-242, Academic Press, New York), the IC₅₀ value for Trolox, a man-made water-soluble Vitamin E commonly used in laboratories, was approximately 16 μM, and the IC₅₀ values for chlorophyll a, chlorophyll b, Compounds 1., 2, 5, 6, 3, and 4 were 5, 26, 10, 42, 17, 54, 21, and 20 μM, respectively. Unexpectedly, all test compounds showed similar antioxidative activity as that of Trolox at a concentration less than 5 μM.

The compounds were also tested in four other assays, i.e., DPPH scavenging assay, superoxide anion scavenging assay, Fe²⁺ chelation assay, and reduction assay. See Shimada et al. J. Agric. Food Chem. (1992) 40:945-948; Robak et al. Biochem. Pharnia. (1988) 37:837-841; Dinis et al. Arch. Biochem. Biophys. (1994) 315:161-169; and Oyaizu et al. Nippon Shokuhin Kogyo Gakkaishi (1986) 35:771-775. Compounds 3 and 4 were stronger scavengers than Compounds 5 and 6 in removing DPPH, which in turn were stronger scavengers than chlorophylls a and b and Compounds 1 and 2. The IC₅₀ values for Compound 4, Compound 3, Compound 6, Compound 5, chlorophyll b, chlorophyll a, and Compound 2 were approximately 28, 40, 120, 160, 200, 265, and 535 μM, respectively, and that for Compound 1 was greater than 700 μM. For scavenging superoxide anion, Compounds 1-4 exhibited an initial increase followed by a decrease in their scavenging ability as their concentrations increased, whereas chlorophylls and Compounds 5 and 6 showed a gradual increase and a subsequent plateau in their scavenging ability. Fe²⁺ chelating efficiencies of the test compounds showed no difference from those determined by other testing systems. All test compounds showed similar chelating strengths. The I₅₀ values as to chelating Fe²⁺ were lower than those as to scavenging DPPH free radical. While most of the test compounds exhibited strong antioxidative capacities, they showed relatively weak reducing power, as compared to Trolox.

Anti-DNA Oxidative Damages

Human lymphocytes were isolated from blood samples of ten healthy men at the age of 25-45 by gradient centrifugation. Cell viability was determined using the MTS (i.e., 3-(4,5-dimethylthiazol-2-yl)-5-(3-carboxymethoxyphenyl)-2-(4-sulfophenyl)-2H-tetrazolium) assay. Compounds 1-4, as well as chlorophyllin, were tested. Lymphocytes were incubated with each compound for 30 min and then exposed to H₂O₂ solutions at concentrations of 10 μM and 50 μM for 5 min at 37° C. The comet assay was then used to detect DNA oxidative damage as described in e.g., Olive et al. Radiation Research (1990) 122:86-94 and Singh et al. Exp. Cell Res. (1988) 175:184-191.

The results show that Compounds 1-4 and chlorophyllin reduced DNA damage on human lymphocytes induced by exposure to a 10 μM H₂O₂ solution. Compounds 3-4 also reduced DNA damage on human lymphocytes induced by exposure to a 50 μM H₂O₂ solution.

### OTHER EMBODIMENTS

All of the features disclosed in this specification may be combined in any combination. Each feature-disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous a compound described in the specification also can be made, and used to practice this invention. Thus, other embodiments are also within the claims.

What is claimed is:

1. A method for eliciting an antioxidative effect, comprising administering to a subject in need thereof an effective amount of a tetrapyrrole compound of formula (I): 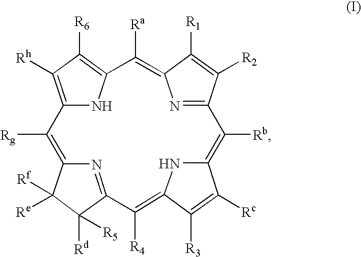
wherein
each of R^{a}, R^{b}, R^{c}, R^{d}, R^{e}, R^{f}, R^{g}, and R^{h}, independently, is H or alkyl;
R₁ is alkyl or CHO;
R₂ is alkyl;
R₃ and R₄ taken together are part of a cyclyl or heterocyclyl ring, and R₅ is (CH₂)ₘCOOR, in which R is H, and m is 1, 2, or 3; or R₃ is (CH₂)ₙCOOR′, C(O)(CH₂)ₙCOOR′, CH(OH)(CH₂)ₙCOOR′, and R₄ and R₅ taken together are part of a cyclyl or heterocyclyl ring, in which R′ is H or alkyl, and n is 0, 1, 2, or 3; and
R₆ is alkenyl or CHO;
the tetrapyrrole compound optionally being chelated with Mg²⁺, Mn²⁺, Cu²⁺, Fe²⁺, Co²⁺, Ni²⁺, or Zn²⁺ through the nitrogen atoms on the four pyrrole rings.

2. The method of claim 1, wherein each of R^{a}, R^{b}, R^{d}, R^{e}, and R^{g} is H; and each of R^{c}, R^{f} and R^{h} is CH₃.

3. The method of claim 1, wherein the tetrapyrrole compound is chelated with Mg²⁺ through the nitrogen atoms on the four pyrrole rings.

4. The method of claim 1, wherein R₂ is CH₂CH₃.

5. The method of claim 4, wherein R₁ is CH₃ or CHO.

6. The method of claim 1, wherein R₃ and R₄ taken together are

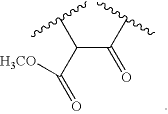

7. The method of claim 1, wherein R₅ is CH₂CH₂COOH, and R₆ is CH═CH₂.

8. The method of claim 7, wherein each of R^{a}, R^{b}, R^{d}, R^{e}, and R^{g} is H; and each of R^{c}, R^{f} and R^{h} is CH₃.

9. The method of claim 7, wherein the tetrapyrrole compound is chelated with Mg²⁺ through the nitrogen atoms on the four pyrrole rings.

10. The method of claim 7, wherein R₂ is CH₂CH₃.

11. The method of claim 10, wherein R₁ is CH₃ or CHO.

12. The method of claim 7, wherein R₃ and R₄ taken together are

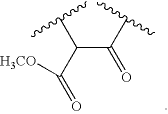

13. The method of claim 12, wherein each of R^{a}, R^{b}, R^{d}, R^{e}, and R_{g} is H; and each of R^{c}, R^{f} and R^{h} is CH₃.

14. The method of claim 13, wherein the tetrapyrrole compound is chelated with Mg²⁺ through the nitrogen atoms on the four pyrrole rings.

15. The method of claim 14, wherein R₂ is CH₂CH₃.

16. The method of claim 15, wherein R₁ is CH₃ or CHO.

17. A method for treating cancer, comprising administering to a subject in need thereof an effective amount of a tetrapyrrole compound of formula (I): 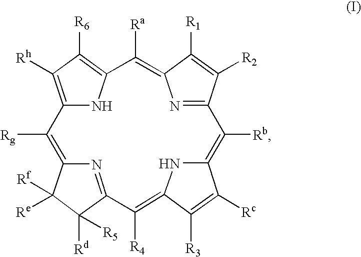
wherein
each of R^{a}, R^{b}, R^{c}, R^{d}, R^{e}, R^{f}, R^{g}, and R^{h}, independently, is H or alkyl;
R₁ is alkyl or CHO;
R₂ is alkyl;
R₃ and R₄ taken together are part of a cyclyl or heterocyclyl ring, and R₅ is (CH₂)ₘCOOR, in which R is H, or [CH₂—CH═C(CH₃)—CH₂]ₘ₁—[CH₂—CH₂—CH(CH₃)—CH₂]ₘ₂—[CH₂CH═C(CH₃)—CH₂]ₘ₃—[CH₂—CH₂—CH(CH₃)—CH₂]ₘ₄—H; m is 1, 2, or 3, and each of m1, m2, m3, and m4, independently, is 0, 1, 2, 3, 4, or 5; or R₃ is (CH₂)ₙCOOR′, C(O)(CH₂)ₙCOOR′, CH(OH)(CH₂)ₙCOOR′, and R₄ and R₅ taken together are part of a cyclyl or heterocyclyl ring, in which R′ is H or alkyl, and n is 0, 1, 2, or 3; and
R₆ is alkenyl or CHO;
provided that if R is H, the tetrapyrrole compound optionally is chelated with Mg²⁺, Mn²⁺, Cu²⁺, Fe²⁺, Co²⁺, Ni²⁺, or Zn²⁺ through the nitrogen atoms on the four pyrrole rings.

18. The method of claim 17, wherein R₅ is (CH₂)ₘCOO[CH₂—CH—C(CH₃)—CH₂]ₘ₁—[CH₂CH₂—CH(CH₃)—CH₂]ₘ₂[—CH₂—CH═C(CH₃)—CH₂]ₘ₃—[CH₂—CH₂—CH(CH₃)—CH₂]ₘ₄—H.

19. The method of claim 18, wherein m is 2.

20. The method of claim 19, wherein m1 is 1, m2 is 3, and each of m3 and m4 is 0.

21. The method of claim 20, wherein each of R^{a}, R^{b}, R^{d}, R^{e}, and R^{g} is H; and each of R^{c}, R^{f} and R^{h} is CH₃.

22. The method of claim 21, wherein R₂ is CH₂CH₃.

23. The method of claim 22, wherein R₁ is CH₃ or CHO.

24. The method of claim 23, wherein R₃ and R₄ taken together are

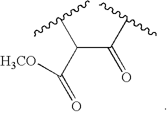

25. The method of claim 24, wherein R₆ is CH═CH₂.

26. The method of claim 18, each of R^{a}, R^{b}, R^{d}, R^{e}, and R^{g} is H; and each of R^{c}, R^{f} and R^{h} is CH₃.

27. The method of claim 18, wherein R₂ is CH₂CH₃, and R₁ is CH₃ or CHO.

28. The method of claim 18, wherein R₃ and R₄ taken together are

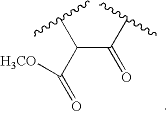

29. The method of claim 17, wherein R₅ is (CH₂)ₘCOOH.

30. The method of claim 29, wherein m is 2.

31. The method of claim 30, wherein the tetrapyrrole compound is chelated with Mg²⁺ through the nitrogen atoms on the four pyrrole rings.

32. The method of claim 31, wherein each of R^{a}, R^{b}, R^{d}, R^{e}, and R^{g} is H; and each of R^{c}, R^{f} and R^{h} is CH₃.

33. The method of claim 32, wherein R₂ is CH₂CH₃.

34. The method of claim 33, wherein R₁ is CH₃ or CHO.

35. The method of claim 34, wherein R₃ and R₄ taken together are

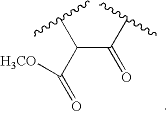

36. The method of claim 35, wherein R₆ is CH═CH₂.

37. The method of claim 29, wherein the tetrapyrrole compound is chelated with Mg²⁺ through the nitrogen atoms on the four pyrrole rings.

38. The method of claim 29, each of R^{a}, R^{b}, R^{d}, R^{e}, and R^{g} is H; and each of R^{c}, R^{f} and R^{h} is CH₃.

39. The method of claim 29, wherein R₂ is CH₂CH₃, and R₁ is CH₃ or CHO.

40. The method of claim 29, wherein R₃ and R₄ taken together are [chemical structure]
